(19) United States
(12) Patent Application Publication
(10) Pub. No.: US 2021/0100857 A1
(43) Pub. Date: Apr. 8, 2021

(54) HERPES 1 ANTIBODY SYSTEM

(71) Applicant: Raymond Joel Rairie, Manchester, NY (US)

(72) Inventor: Raymond Joel Rairie, Manchester, NY (US)

(21) Appl. No.: 17/063,706

(22) Filed: Oct. 5, 2020

Related U.S. Application Data
(63) Continuation-in-part of application No. 15/663,786, filed on Jul. 30, 2017, now abandoned.

(51) Int. Cl. A61K 35/763; C12Q 1/6827

(52) U.S. Cl. CPC A61K 35/763; C07K 16/087; C12Q 1/6827

(57) ABSTRACT

The PINBALL HERPES ANTIBODY SYSTEM prevents people from getting HERPES. It prevents people from getting HERPES SIMPLEX 1 VIRUS (Hsv-1) (HERPES 1) on their genitals, mouth, anus, hands, eyes and face. It also prevents people from getting SIMPLEX 2 VIRUS (Hsv-2) (HERPES 2) on their genitals, mouth, anus, hands, eyes and face. And it prevents a baby from getting infected with HERPES 1 and HERPES 2 during birth.

This application is a Continuation-In-Part of application Ser. No. 15/663,786, filed on Jul. 30, 2017, which is incorporated by reference.

### FIELD

This application is designed to: (1) generate a safe process that will protect people from getting infected with HERPES SIMPLEX 1 (HSV-1)—commonly known as “HERPES 1”—on or around their genitals, mouth, and anus. 100021 And (2) to protect people from getting infected with HERPES SIMPLEX 2 (HSV-2)—commonly known as “HERPES 2” or around their genitals, mouth, and anus. 100031 And (3) prevent babies from getting infected with HERPES 1 and/or HERPES 2 during birth (the baby could die if the baby gets inlècted). 100041 The PINBALL HERPES ANTIBODY SYSTEM involves the composition of matter.

The matter being antibodies for both HERPES 1 and HERPES 2.

### BACKGROUND

The purpose of this patent is to benefit humanity and society and civilization. People enjoy sex very much. Unfortunately—sometimes people contract Herpes on their private parts or mouth. Millions of people have Herpes on their private parts and mouth. Some people have a natural ability to fight off Herpes—this is good for them. Others are able to take medication to control outbreaks. Unfortunately, Herpes medication does not always work—or only works to a certain point. Unfortunately, some people have painful outbreaks on their private areas and mouth two or three or more times per year—for all their life. They lose work. And (unfortunately) sometimes (unintentionally) spread Herpes to their spouse or girlfriend or boyfriend. Herpes can (unfortunately) cause emotional pain. Also, it is very sad to say that Herpes can hurt or kill a baby—sometimes when a mother is giving birth—and the mother has Herpes—the baby can contract. Herpes during the birth. Because the baby is so young—the baby cannot fight off the infection. I would like to fix these sad issues.

### SUMMARY

MY system will intentionally inlèct a person with. Herpes Simplex 1 1) and Herpes Simplex 2 2). The infection shall be controlled and regulated. My system will also generate antibodies usv-1 (HERPES 1) and uSV-2 (HERPES 2). My system will prevent people from getting HERPES I and HERPES 2 on their genitals and other sensitive areas, and prevent people from getting HERPES 1 and HERPES 2 on their mouths. My system will also prevent babies from getting inlècted with HERPES I during birth and prevent babies from getting inlècted with HERPES 2 during birth.

Till/PINBALL HERPES ANTIBODY SYSTEM (THE SYSTEM) is a unique, complicated, time consuming, and expensive process that generates matter (HERPES I antibodies and HERPES 2 antibodies).

THE PINBALL HERPES ANTIBODY SYSTEM will not help people who are already inlècled with HERPES I and HERPES 2. someone is only with HERPES I then they could benefit from THE SYSTEM—they could be protected from HERPES 2. If someone is only inlècled with HERPES 2 then they could benefit from THE SYSTEM they could be protected from HERPES 1.

THE PINBALL HERPES ANTIBODY SYSTEM is the only SYSTEM on the planet Earth that does these things.

THE PINBALL HERPES ANTIBODY SYSTEM will benefit humanity, society, and civilization. It will prevent millions of people, over thousands of years from getting terrible and painful sores on their private areas. And it will save babies THANK YOU.

### BRIEF DESCRIPTION OF THE DRAWING

FIG. 1 is a flowchart showing the steps in the process of the preferred embodiment of the invention.

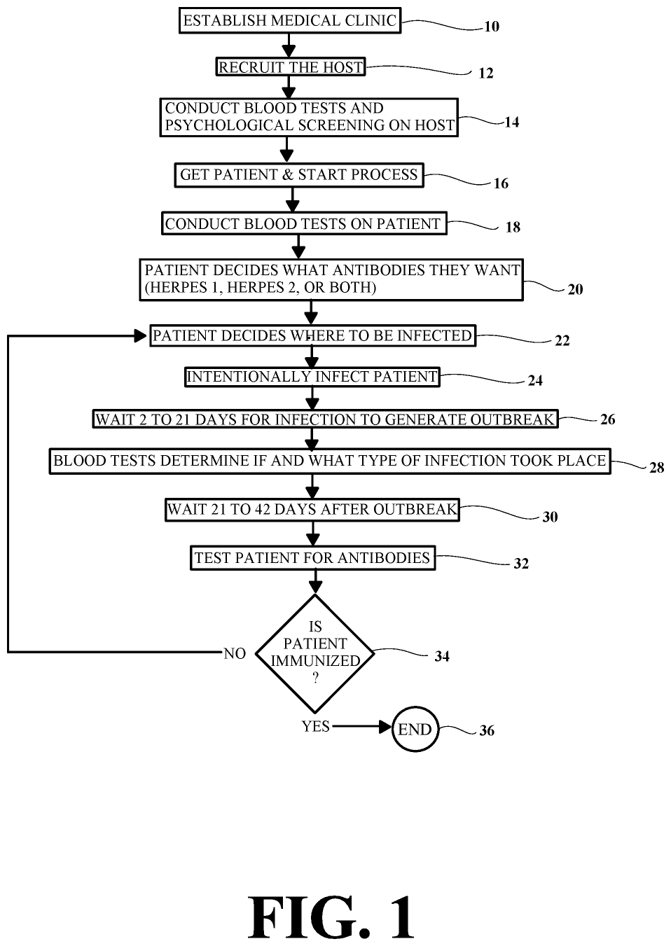

### DESCRIPTION

THE PINBALL HERPES ANTIBODY SYSTEM (hereafter referred to as “THE SYSTEM”) is a [symbol] highly regulated and controlled process. THE SYSTEM is designed to protect the PATIENT (described below) from the diseases of HERPES SIMPLEX 1 HERPES 1) and/or HERPES SIMPLEX 2 HERPES 2). And also to protect a baby during birth from getting HERPES.

THE SYSTEM is unique, complicated, involved, time consuming, and expensive.

### The Medical Clinic

THE SYSTEM shall consist of a professional medical clinic (hereafter referred to as “the clinic”). The clinic shall be under the direct supervision of one or more licensed Medical Doctor(s). The doctor(s) shall have both training and experience in dealing with the disease(s) of HERPES [symbol] SIMPLEX 1 HERPES 1) and HERPES SIMPLEX 2 HERPES 2). The clinic will also employ a professional HUMAN RESOURCE worker—to handle recruitment, direct control, and supervision of the HOST—(the HOST will be described below).

The medical clinic shall have the capacity to take blood samples from the HOSTS and the PATIENTS A licensed nurse(s) shall obtain blood samples from the HOST and the PATIENT—(the PATIENT shall be described below).

### The Patient

There will be two types of people who will utilize the clinic. One type of person will be the person who is currently not inlècted with HERPES I and/or not inlècted with HERPES 2. This type of person will be known as “The IENT”. The PATIENT is paying the clinic to provide The PAVIF,NT with the PINBALL HERPES ANTI-BODY .SYSTEM. The other person who will utilize the clinic will be a person who is already inlècled with HERPES I (I ISV1) or HERPES 2 (IISV-2) this already person shall be known as “the HOST”. The HOST is paid to provide HERPES I and HERPES 2 10 the clinic. The HOST will be subjected to extensive screening (Or their past behavior, and must pass a physical exam, and psychological exam, and health tests (as described below). The HOST is financially compensated (Or their time and ellòrt. The HOST is treated with great respect by THE SYSTEM. Finding the correct HOST is a key part 10 THE SYSTF,Pvl il can be compared .10 finding the correct machine in order lòr a Factory 10 work properly (like a cog in the machinery). Finding the proper HOST could be very difficult and expensive. The HOST must have a certain background in order Ibr THE SYSTEM 10 work correctly. Everything must be put together just right—or THE PINBALL HERPES ANTIBODY SYSTEM will not work correctly.

The PATIENT will be intentionally infected with HERPES 1 and/or HERPES 2. The intentional HERPES infection shall be regulated and controlled (as described below). Licensed medical doctor(s) and licensed nurses shall handle and supervise the process.

### The Host

Selecting the correct HOST is a key part to the PINBALL HERPES ANTIBODY SYSTEM. The HOST will be providing the clinic with HERPES 1 and HERPES 2 samples. The HOST will be tested HERPES 1 and HERPES 2, and the AIDS virus. The HOST will be tested for the Hepatitis B virus. The HOST will be tested for the Hepatitis C virus. The HOST will be tested for Syphilis. The licensed nurse shall take the blood samples of the HOST at the clinic. The blood samples shall be subject to the latest, most reliable, and most sophisticated testing possible. The HUMAN RESOURCE worker will recruit the HOST. The HOST will be subject to questions regarding their personal behavior—such as illegal drug use and sexual activity. The HOST will be subject to psychological testing, screening, and background checks. The HOST shall be treated with the greatest respect—in a way the host will be a VERY IMPORTANT PERSON (V.I.P.). A good comparison could be having the correct quarterback on a football team—or else it does not matter if you have other good players on the team. [00191 The reason the HOST is subject to these tests and procedures—is that the HOST will be providing the HERPES samples from visible HERPES sores on the HOST'S lips. The transfer shall take place with a swab (as described below). The HOST must be in the proper physical condition and proper psychological condition to provide the PATIENT with HERPES 1 and/or HERPES 2. This is done to insure the PATIENT does not become infected with AIDS, nor Hepatitis B, nor Hepatitis C, nor Syphilis. The HOST will be subjected to psychological testing and screening. THE SYSTEM will not work correctly if the HOST does things—such as: injecting heroin the night before they are scheduled to contribute a HERPES sample and then exposing the IENT to AIDS or IIF,PAVITIS B. This is why it is so important to have the professional HUMAN RESOURCE worker as part of Till/PINBALL HERPES ANTIBODY SYSTEM. This part will take considerable time, ellòrt, and money. The HUMAN RESOURCE worker will be responsible [Or the HOST to keep the HOST from doing the wrong things.

Some HOSTS will provide only HERPES 1 samples. Some HOSTS will provide only HERPES 2 samples. Some HOSTS will provide both HERPES 1 samples and HERPES 2 samples. The HUMAN .RESOURCE worker and the DOCTOR(S) will analyze all relevant data to insure a potential HOST has all of the necessary Factors to salèly provide the clinic with HERPES 1 and/or HERPES 2. If this section of THE SYSTEM is not done correctly a patient could become inlècted with AIDS or IIEPAVITIS B or IIEPAVITIS C or Syphilis. The best and most sophisticated and most reliable medical tests will be used 10 determine if the HOST is inlècled with HERPES I and/or HERPES 2 and if they have a current HERPES 1 or HERPES 2 outbreak on their lips.

When a person (the PATIENT) wants 10 gain protection from HERPES I and/or HERPES 2 from infecting their genitals, mouth, anus, hands, face, and eyes—they can make an appointment with the clinic. At the clinic they will be subject to blood tests. The blood tests will be the best, most sophisticated, and most reliable possible. It will be determined if they are already infected with HERPES I or HERPES 2. If they are already infected with HERPES I they will not need THE SYSTEM for HERPES 1. But they could still use THE SYSTEM to get protected from HERPES 2. If they are already infected with HERPES 2 they will not need THE SYSTEM for HERPES 2. But they could still use THE SYSTEM to get protected from HERPES 1.

Once a person is infected with HERPES I—that person will develop HERPES I antibodies. Once a person has HERPES 1 antibodies they can no longer get HERPES 1. (But they could still have outbreaks at the original HERPES 1 infection area).

Once a person is infected with HERPES 2—that person will develop HERPES 2 antibodies. Once a person has HERPES 2 antibodies they can no longer get HERPES 2. (But they could still have outbreaks at the original HERPES 2 infection area).

If the PATIENT is not infected with HERPES 1 nor infected with HERPES 2: then THE PINBALL HERPES ANTIBODY SYSTEM will prevent the PATIENT from getting HERPES 1 and HERPES 2 on their genitals. And prevent the PATIENT from getting both HERPES 1 and HERPES 2 on or in their mouth. And prevent the PATIENT from getting both HERPES 1 and HERPES 2 on and inside their anus, also protecting their hands, face, and eyes.

If the PATIENT is already infected with HERPES

1—then THE SYSTEM will not protect them from HERPES 1. But if the PATIENT is not infected with HERPES 2—then THE SYSTEM will protect them from HERPES 2.

If the PATIENT is already infected with HERPES

2—then THE SYSTEM will not protect them from HERPES 2. But if the PATIENT is not infected with HERPES I—then THE SYSTEM will protect them from HERPES 1.

Blood tests at the clinic will determine if the PATIENT has already been infected with HERPES 1 and/or HERPES 2. If the PATIENT has already been with both HERPES 1 and HERPES 2 then THE SYSTEM cannot help them.

If after the original blood tests it is determined that a PAVIF,NT can benefit from Till/SYSTEM: then the PAI′IF,NT is subject to the next step in the process.

The PAI′IF,NT will be inlòrmed of the next step in Till/SYSTEM. The PATIENT will be intentionally infected with: HERPES I1 only; or HERPES 2 only; or both HERPES 1 and HERPES 2. The PATIENT will decide on what type of inlèction they will get after discussing the situation with the doctor(s).

The intentional inlèction process will take place at a location or locations on the PATIENT'S body that will not be as sensitive as their genitals, nor mouth, nor anus (which are normal areas Ibr HERPES inlèclion).

### The Swabbing

The intentional HERPES inlèction shall be at a location on the PATIENT'S body the PATIENT has selected 10 be intentionally inlècled. The PAI′IF,NT shall discuss the situation with the doctor(s) And then make a .decision. If the PAI′IF,NT is only going 10 gel one intentional HERPES inlèclion then only one location will be selected .by the PATIENT. If the PATIENT is 10 gel two intentional HERPES infections—then the PATIENT shall select two areas on their body to be infected. The PATIENT shall have full authority on where the intentional HERPES infection shall be. If the PATIENT is intentionally getting infected with both HERPES 1 and HERPES 2 the PATIENT will make the decision for both locations. Possible non-sensitive locations could be under the upper arm—by the armpit or on the top of a foot. Or some other area of the body—as determined by the PATIENT (after discussing the situation with the doctor(s)).

The area(s) of intentional HERPES infection shall be subjected to a medically professional incision made by the doctor(s) at the clinic. The equipment used for the incision shall be medically professional and medically sterile. The intentional infection shall be done by the doctor(s) or licensed nurse—using a swab. The doctor(s) shall determine the best material possible to make and use for the swab. The swab shall be medically professionally made and medically professionally disinfected and medically professionally clean—and kept in a medically professional sterile package. And kept in the medically professional sterile package until ready for immediate use.

The doctor(s) or licensed nurse shall take the sterile swab and use the sterile swab to make contact with the HOST'S visible HERPES 1 sore(s) on the HOST'S lips. And then intentionally transfer the HERPES I virus from the HOST'S lips to the PATIENT'S intentional incision for the HERPES I intentional infection.

The doctor(s) or licensed nurse shall take the sterile swab and use the sterile swab to make contact with the HOST'S visible HERPES 2 sore(s) on the HOST'S lips. And then intentionally transfer the HERPES 2 virus from the HOST'S lips to the PATIENT'S intentional incision for the HERPES 2 intentional infection.

If the PATIENT is only getting infected with HERPES I—then only one incision will be made. If the PATIENT is only getting infected with HERPES 2—then only one incision will be made. If the PATIENT is getting infected with both HERPES 1 and HERPES 2—then the doctor(s) will make two incisions. This is done to see if and when an original HERPES outbreak has occurred. One location will be (Or HERPES I and the other location will be for HERPES 2. The doctor(s) will keep a record of where each and what type of intentional inlèction took place. This will help the doctor(s) determine if an inlèction actually took place (as described below)—or whether the intentional inlèction process (the swabbing process) needs to be repeated. And if it needs to be repeated the doctor(s) will know which inlèction needs to be I or HERPES 2). Also, sophisticated, expensive, and reliable blood tests will be done about 21 to 42 days after the original outbreak both HERPES 1 and HERPES 2 to determine if HERPES antibodies have been generated by the PATIENT'S body. And exactly which HERPES antibodies have been generated (HERPES I or HERPES 2 or both).

### Inlèclion

A typical HERPES I inlèction will occur from 2 days 10 21 days after original inlèclion. After 21 days the doctor(s) will conduct the most reliable, most modern, sophisticated, and expensive blood test(s) to determine if any and what type of HERPES inlèclion(s) look place. If the IENT does not have a visible HERPES outbreak alter 21 days then it is possible that the PAI′IF,NT will not have a visible outbreak. But, the patient could still have been infected (meaning the intentional swabbing was effective). The blood tests will provide the doctor(s) with the information needed to determine whether or not there was a HERPES I infection or a HERPES 2 infection or no infection at all. The doctor(s) will then wait another 21 to 42 days to conduct new sophisticated, most reliable, most modern, and expensive blood tests to determine whether or not HERPES I antibodies have been generated by the PATIENT.

If HERPES I antibodies have been detected (by the blood test(s))—then THE SYSTEM worked for HERPES 1. 100381 If HERPES I antibodies have not been detected—then the swabbing did not generate a HERPES I inlèction. The process is repeated Iòr HERPES 1.

Once the PATIENT has antibodies for HERPES I—then the PATIENT is immuned to getting a HERPES 1 infection anywhere else on their body.

Once antibodies for HERPES I are generated by a person's body—the antibodies for HERPES I are generated for the life of that person.

A typical HERPES 2 infection will occur from 2 days to 21 days after original infection. After 21 days the doctor(s) will conduct the most reliable, most modern, expensive, and sophisticated blood test(s) to determine if any and what type of HERPES infection(s) took place. If the PATIENT does not have a visible HERPES outbreak after 21 days—then it is possible that the PATIENT will not have a visible outbreak. But, the patient could still have been infected (meaning the intentional swabbing was effective). The blood tests will provide the doctors with the information needed to determine whether or not there was a HERPES I infection or a HERPES 2 infection or no infection at all. The doctor(s) will then wait another 21 to 42 days to conduct new sophisticated, most reliable, most modern, and expensive blood tests to determine whether or not HERPES 2 antibodies have been generated by the PATIENT.

If HERPES 2 antibodies have been detected (by the blood test(s))—then THE SYSTEM worked for HERPES 2. 100431 If HERPES 2 antibodies have not been detected—then the swabbing did not generate a HERPES 2 infection. The process is repeated (Or HERPES 2.

Once the PATIENT has antibodies for HERPES 2—then the PATIENT is immuned to getting a HERPES 2 inlèction anywhere else on their body.

Once antibodies Iòr HERPES 2 are generated by a person's body the antibodies [Or HERPES 2 are generated Ibr the lilè of that person.

### Protection [Or a Baby

If a mother has generated antibodies (Or both HERPES 1 and HERPES 2 with the PINBALL HERPES ANTIBODY SYSTEM: then the mother does not need to worry about getting HERPES 1 nor HERPES 2. so there is now no chance of her baby getting HERPES during birth.

### FLOWCHART FOR THE PINBALL HERPES ANTIBODY SYSTEM

ESTABLISH MEDICAL CLINIC

RECRUIT THE HOST

CONDUCT BLOOD TESTS and PSYCHOLOGICAL SCREENING ON HOST

GET PATIENT—START PROCESS

CONDUCT BLOOD TESTS ON PATIENT

PATIENT DECIDES WHAT ANTIBODIES DO THEY WANT (HERPES 1 or HERPES 2 or BOTH)

PATIENT DECIDES WHERE TO BE INFECTED

INTENTIONALLY INFECT PATIENT

WAIT 2 to 21 DAYS for INFECTION TO GENERATE (OUTBREAK)

BLOOD TESTS TO DETERMINE IF and WHAT TYPE OF INFECTION TOOK PLACE

WAIT 21 to 42 DAYS AFTER OUTBREAK

TEST PATIENT FOR ANTIBODIES

PATIENT IMMUNF,D or REPEAT PROCESS

I claim:

1. A process for creating immunity to Herpes Simplex Type 1, comprising the steps of:
obtaining a limited number of Herpes Simplex Virus One (HSV-1) viruses from a first person (“Host”);
transferring said unmodified viruses to a second person (“Patient”) to deliberately infect said Patient and induce said Patient's immune system to create antibodies that will make said Patient immune to Herpes Simplex Virus One (HSV-1) infection on additional, separate, and different locations of said Patient's body; and
testing said Patient for one or more genes that increase the risk an HSV-1 infection will cause said Patient to develop a second disease, and to reduce the probability of said Patient developing a third disease.

2. The process according to claim 1, wherein:
the one or more genes include the Apolipoprotein 4 (“Apoe-4”) gene, and the second disease is selected from the group comprising Dementia and Alzheimer's disease. And, said third disease is Herpes Simplex Encephalitis (HSE). If a human being has the Apolipoprotein 4 (“Apoe-4”) gene, and the same human being has said HSV-1 in their brain—then the probability of said human being developing and/or contracting Dementia and/or Alzheimer's disease increases. By identifying said gene, said Patient understands, that by reducing the probability that HSV-1 enters said Patient's brain: said Patient is reducing the probability that said Patient will develop and/or contract Dementia and/or Alzheimer's disease.

3. The process according to claim 1, wherein:
reduces the risk that HSV-1 will infect the Trigeminal Ganglia. Said process will place the HSV-1 in said Patient's Sacral Ganglia. The Sacral Ganglia is in the lower part of said Patient's body. The Trigeminal Ganglia is located in the upper part of said Patient's body—(near the brain). Said Patient is intentionally infected with an HSV-1 viral load of between 10 to the 4th power of HSV-1 Deoxyribonucleic Acid (DNA) copies and 10 to the 7th power of HSV-1 Deoxyribonucleic Acid (DNA) copies.

4. The process according to claim 1, wherein:
said process takes place in a medical clinic under professional medical supervision;
said Patient pays the clinic to provide said process;
said Host is selected by the clinic from persons already having HSV-1;
said Host is paid by the clinic to provide HSV-1 viruses;
said Host provides blood samples;
said Host is tested for HSV-1, said Host must currently have an active HSV-1 outbreak.
Said HSV-1 may be medically stored for future use.
Said HSV-1 may be medically analyzed by HSV-1 sub-type strain.
Said HSV-1 shall be negative for any pathogens.
said Host is tested for (and must be negative for): Herpes Simplex Virus Two (HSV-2), AIDS, Hepatitis B, Hepatitis C, and Syphilis;
said Host is questioned and tested regarding sexual activity and recreational drug use;
said Host is subjected to psychological testing and background checks;
said Host is recruited and supervised by a human resource worker;
before said viruses are transferred, said Patient is tested for HSV-1, and if said Patient already has HSV-1 antibodies, the transfer is not made;

5. The process according to claim 1, wherein:
The intentional infection area (incision) shall be selected by said Patient at the upper back of the right leg, or the upper back of the left leg, or some other area of said Patient's body that is connected to said Patient's Sacral Ganglia, and said HSV-1 infection shall go dormant in said Sacral Ganglia. Because said HSV-1 goes dormant in said Sacral Ganglia, there is a significant reduction in the probability of HSV-1 going dormant in said Patient's Trigeminal Ganglia. Said HSV-1 are transferred to an area on said Patient's body that is/are less sensitive than said Patient's mouth or genitals. By intentionally infecting a specific area of said Patient's body with HSV-1: said Pinball System will control where the original HSV-1 outbreak will occur, and also there will be a very high probability that all recurrent HSV-1 outbreaks will be at the same (original) location.

6. The process according to claim 1, wherein:
a professional medical sterile swab is used to transfer said HSV-1 viruses;
the swab is kept in a sterile package before use;
said incision is made in the skin in the area of said Patient's body so HSV-1 viruses are transferred; and
said incision is made by a licensed health care professional.

7. The process according to claim 1, wherein: said Patient's blood is tested for antibodies to HSV-1 after the viruses are transferred; said Patient's blood is tested for antibodies to HSV-1; 21 to 42 days after an original outbreak following transfer of said HSV-1 viruses; said process is repeated if no antibodies for HSV-1 are found; if there is no visible outbreak, said Patient's blood is tested for antibodies to HSV-1; 21 to 42 days after transfer of said HSV-1; said process is repeated if no HSV-1 antibodies are found; and once antibodies to said HSV-1 are found in said Patient's blood, said Patient is told that he or she is now immune to HSV-1 infection in said Patient's mouth and genitals.

8. The process according to claim 8, wherein: once HSV-1 antibodies are found in said Patient's blood, said Patient is immune from contracting HSV-1 on their mouth and genitals. Said Patient now has a reduction in the probability of HSV-1 entering said Patient's Trigeminal Ganglia.

9. The process according to claim 8, wherein: once antibodies to HSV-1 are found in said Patient's blood, if said Patient is a woman, said woman is prevented from passing said HSV-1 to her baby when said baby is born—and thus preventing Neonatal Herpes baby blindness, and Neonatal Herpes Encephalitis, and Neonatal Herpes baby death.

10. The process according to claim 1, wherein: because of how the Pinball System transfers and manipulates the HSV-1 there is a difference between how HSV-1 effects the first person compared to how HSV-1 effects the second person. HSV-1 in the first person is harmful, but because of the Pinball System, HSV-1 has a reduction in the probability of being harmful to the second person. This is a fundamental difference between the first person and the second person.

11. A composition of matter, comprising antibodies produced by: obtaining a limited number of Herpes Simplex Virus One (HSV-1) from a first person (“Host”); transferring the unmodified HSV-1 viruses to a second person (“Patient”) to deliberately infect said Patient and induce said Patient's immune system to create the HSV-1 antibodies that will make said Patient immune to Herpes Simplex Virus One (HSV-1); and testing said Patient for one or more genes that increase the risk an HSV-1 infection will cause said Patient to develop a second disease. And, prevent said Patient from developing a third disease.

12. The composition of matter according to claim 11, wherein: the one or more genes include the Apolipoprotein 4 (“Apoe-4”) gene, and the second disease is Dementia-Alzheimer' s Disease. And, the third disease is Herpes Simplex Encephalitis (HSE). If a human being has the Apolipoprotein 4 (“Apoe-4”) gene, and the same human being has said HSV-1 in their brain—then the probability of said human being developing and/or contracting Dementia and/or Alzheimer's disease increases. By identifying said gene, said Patient understands, that by reducing the probability that HSV-1 enters said Patient's brain: said Patient is reducing the probability that said Patient will develop and/or contract Dementia and/or Alzheimer's disease.

13. The composition of matter according to claim 11, wherein: reduces the risk that HSV-1 will infect the Trigeminal Ganglia. The intentional generation of HSV-1 antibodies (composition of matter) will prevent an HSV-1 infection in the face, mouth, and eye areas of said Patient—and therefore there is a reduction in the probability that HSV-1 will enter the Trigeminal Ganglia—and therefore there is a reduction in the probability of HSV-1 entering the brain of said Patient. The Pinball Herpes Antibody System uses the active step of identifying the Apolipoprotein 4 (“Apoe-4”) gene. And, also the active step of intentionally placing said HSV-1 into the Sacral Ganglia. And, also the active step of intentionally preventing said HSV-1 from entering the Trigeminal Ganglia. And, also the active step of regulating the amount of HSV-1 that is necessary to induce the Patient to generate HSV-1 antibodies. And, the active step of reducing the probability that said Patient will develop and/or contract Dementia and/or Alzheimer's Disease. And, the active step of reducing the probability that said HSV-1 will enter said Patient's brain and generate Herpes Simplex Encephalitis (HSE).

14. The composition of matter according to claim 11, wherein: the Pinball System is a distinct process to generate HSV-1 antibodies—because the antibodies that are generated by the Pinball System are intentionally generated to reduce the probability of the Patient developing and/or contracting Dementia and/or Alzheimer's Disease—by reducing the probability of HSV-1 entering the brain. HSV-1 by itself increases the probability of a human being developing and/or contracting Dementia and/or Alzheimer's—but when HSV-1 is subjected to the Pinball System: HSV-1 is manipulated to reduce the probability of a human being developing and/or contracting Dementia and/or Alzheimer's Disease.

15. The composition of matter according to claim 11, wherein:
the composition of matter takes place in a medical clinic under professional supervision;
said patient pays the clinic to provide the composition of matter;
Hosts are selected by the clinic from persons already having HSV-1;
Hosts are paid by the clinic to provide HSV-1;
said Host provides blood samples;
said Host must have a visible HSV-1 outbreak (to use for swabbing);
Said HSV-1 may be medically stored for future use.
Said HSV-1 may be medically analyzed by HSV-1 sub-type strain.
Said HSV-1 shall be negative for any pathogens.
said Host is tested for (and must not be infected with) HSV-2, AIDS, Hepatitis B, Hepatitis C, and Syphilis;
said Host is questioned regarding sexual activity and recreational drug use;
said Host is subjected to psychological testing and background checks;
said Host is recruited and supervised by a human resource worker.

16. The composition of matter according to claim 11, wherein:
Said Patient is intentionally infected with an HSV-1 viral load of between 10 to the 4th power of HSV-1 Deoxyribonucleic Acid (DNA) copies and 10 to the 7th power of HSV-1 Deoxyribonucleic Acid (DNA) copies.
Before the viruses are transferred, said Patient is tested for HSV-1, and if said Patient already has HSV-1, the transfer is not made;
The intentional infection area (incision) shall be selected by said Patient behind the upper right leg, or behind the upper left leg, or some other area of said Patient's body that is connected to said Patient's Sacral Ganglia, and said HSV-1 infection shall go dormant in said Sacral Ganglia. Said intentional infection area shall be less sensitive than their mouth or genitals. By intentionally infecting a specific area of said Patient's body with HSV-1—said Pinball System will control where the original HSV-1 outbreak will occur, and also there will be a very high probability that all recurrent HSV-1 outbreaks will be at the same (original) location.

17. The composition of matter according to claim 11, wherein:
a professional medical swab is used to transfer the viruses;
the swab is kept in a sterile package before use;
an incision is made in the skin in the area of the body to which HSV-1 are transferred: and
the incision is made by a licensed health care professional, at a professional medical facility.

18. The composition of matter according to claim 11, wherein:
said Patient's blood is tested for antibodies to HSV-1 after the HSV-1 are transferred;
said Patient's blood is tested for antibodies to HSV-1; 21 to 42 days after an original outbreak following transfer of said HSV-1;
the process is repeated if no HSV-1 antibodies are found;
if there is no visible outbreak, said Patient's blood is tested for antibodies to HSV-1; 21 to 42 days after transfer of said HSV-1;
the process is repeated if no HSV-1 are found; and
once antibodies to HSV-1 are found in said Patient's blood, said Patient is told that he or she is now immune from contracting HSV-1 on their mouth and genitals.

19. The composition of matter according to claim 18, wherein:
once antibodies to HSV-1 are found in the Patient's blood, said Patient is immune from contracting HSV-1 on their mouth and genitals.

20. The composition of matter according to claim 18, wherein:
once antibodies to HSV-1 are found in said Patient's blood, if said Patient is a woman, she is prevented from passing HSV-1 to her baby when said baby is born (and preventing Neonatal Herpes)—thus saving babies.
